(19) United States
(12) Patent Application Publication
(10) Pub. No.: US 2019/0345517 A1
(43) Pub. Date: Nov. 14, 2019

(54) GENOME EDITING KIT

(71) Applicant: ISHIHARA SANGYO KAISHA, LTD., Osaka (JP)

(72) Inventor: Tomona YAMAGUCHI, Osaka (JP)

(73) Assignee: ISHIHARA SANGYO KAISHA, LTD., Osaka (JP)

(21) Appl. No.: 16/363,398

(22) Filed: Mar. 25, 2019

Related U.S. Application Data
(60) Provisional application No. 62/757,901, filed on Nov. 9, 2018.

(30) Foreign Application Priority Data
May 14, 2018 (JP) 2018-093326

(51) Int. Cl. C12N 15/90; C12N 9/22; C12N 15/113; C07K 14/08; C07K 14/46

(52) U.S. Cl. CPC C12N 15/902; C12N 9/22; C07K 14/461; C07K 14/08; C12N 15/113

(57) ABSTRACT

A genome editing kit includes a viral envelope, a Cas9 protein, a surfactant, and at least one positively charged substance selected from the group consisting of protamine sulfate, polyarginine, polylysine, polyethyleneimine, and hexadimethrine bromide. A genome editing composition includes a viral envelope, a Cas9 protein, a guide RNA, and a positively charged substance. A genome editing method and a method for producing a genome-edited cell or organism include the step of bringing a viral envelope, a Cas9 protein, a guide RNA, and a positively charged substance into contact with a cell or organism. The genome editing kit etc. are capable of editing genomes of cells (e.g., immune cells (strain)) that are conventionally difficult to transfect.

### TECHNICAL FIELD

The present invention relates to a genome editing kit, a genome editing composition, a genome editing method, and a method for producing a genome-edited cell or organism.

### BACKGROUND

Many researchers have recently utilized genome editing technology using the CRISPR/Cas9 system, which is a third-generation genome editing tool following ZFNs and TALENs. This technology is essential for life-science study; however, many cells, such as immune cells, have low genome editing efficiency.

HVJ-E is one of the transfection reagents. HVJ-E is a particle in which a genome is completely inactivated while the cell membrane fusion capability of Sendai virus (a hemagglutinating virus of Japan) is maintained. HVJ-E is also a non-viral transfection tool that is used safely at a general laboratory level, without the need for special operation and facilities. When an HVJ-E vector having DNA, protein, antisense oligonucleotide, siRNA/miRNA, etc., encapsulated therein comes into contact with a target cell, NH protein on the surface of the envelope binds to a sialic acid on the membrane of the target cell; and F protein causes membrane fusion, thus introducing an encapsulated molecule into the target cell.

As the technique using such HVJ-E, JP2005-343874A, for example, has reported that a foreign substance can be introduced with high efficiency by bringing an inactivated viral envelope into contact with a surfactant before the step of mixing the inactivated viral envelope and the foreign substance. JP2005-343874A has also reported that since centrifugation is not performed after the inactivated envelope comes into contact with the foreign substance, operation properties are improved, thus facilitating the preparation of a composition for introducing a foreign substance.

JP2008-212023A has reported the following. A surfactant, protamine sulfate, and protein are added to a solution containing HVJ-E. The solution is stirred to thereby encapsulate an HVJ-E protein therein, and then brought into contact with cells. The protein can thus be easily and efficiently introduced into the cells merely by mixing the HVJ-E and protein in a single tube.

WO2015/005431 has reported that a composition for introducing a gene into a cell with high safety and high gene introduction efficiency can be obtained by a simple operation of merely mixing (A) a lipid such as sorbitan sesquioleate, (B) a protein such as albumin, and (C) a positively charged substance such as protamine sulfate. PTL 3 has also reported that the composition for gene introduction may further contain (F) a viral envelope such as HVJ.

### SUMMARY

The present invention provides, as shown below, a genome editing kit, a genome editing composition, a genome editing method, and a method for producing a genome-edited cell or organism.

### (I) Genome Editing Kit

(I-1) A genome editing kit comprising a viral envelope, a Cas9 protein, a surfactant, and at least one positively charged substance selected from the group consisting of protamine sulfate, polyarginine, polylysine, polyethyleneimine, and hexadimethrine bromide.
(I-2) The kit according to Item (I-1), wherein the positively charged substance is protamine sulfate.
(I-3) The kit according to Item (I-1) or (I-2), which further comprises a guide RNA.
(I-4) The kit according to any one of Items (I-1) to (I-3), which further comprises a donor DNA.

### (II) Genome Editing Composition

(II-1) A genome editing composition comprising a viral envelope, a Cas9 protein, a guide RNA, and at least one positively charged substance selected from, the group consisting of protamine sulfate, polyarginine, polylysine, polyethyleneimine, and hexadimethrine bromide.
(II-2) The composition according to Item (II-1), which further comprises a donor DNA.
(II-3) The composition according to Item (II-1) or (II-2), wherein the positively charged substance is protamine sulfate.

### (III) Genome Editing Method

(III-1) A genome editing method comprising the step of bringing a viral envelope, a Cas9 protein, a guide RNA, and at least one positively charged substance selected from the group consisting of protamine sulfate, polyarginine, polylysine, polyethyleneimine, and hexadimethrine bromide into contact with a cell or organism.
(III-2) The method according to Item (III-1), comprising the steps of:
(1) bringing the viral envelope into contact with the Cas9 protein,
(2) bringing the mixture comprising the viral envelope and the Cas9 protein obtained in step (1) into contact with the surfactant,
(3) bringing the mixture comprising the viral envelope and the Cas9 protein obtained in step (2) into contact with the guide RNA,
(4) bringing the mixture comprising the viral envelope, the Cas9 protein, and the guide RNA obtained in step (3) into contact with at least one positively charged substance selected from the group consisting of protamine sulfate, polyarginine, polylysine, polyethyleneimine, and hexadimethrine bromide, and
(5) bringing the mixture comprising the viral envelope, the Cas9 protein, the guide RNA, and the positively charged substance obtained in step (4) into contact with the cell or organism.
(III-3) The method according to Item (III-1), wherein a donor DNA is further brought into contact with the cell or organism in the step.
(III-4) The method according to Item (III-2), wherein the mixture comprising the Cas9 protein, the viral envelope and the guide RNA is further brought into contact with a donor DNA in step (3).
(III-5) The method according to any one of Items (III-1) to (III-4), wherein the organism is a non-human organism.
(III-6) The method according to any one of Items (III-1) to (III-4), wherein the cell or organism is a suspension cell.
(III-7) The method according to any one of Items (III-1) to (III-6), wherein the positively charged substance is protamine sulfate.

### (IV) Method for Producing Genome-Edited Cell or Organism

(IV-1) A method for producing a genome-edited cell or organism, the method comprising the step of bringing a envelope, a Cas9 protein, a guide RNA, and at least one positively charged substance selected from the group consisting of protamine sulfate, polyarginine, polylysine, polyethyleneimine, and hexadimethrine bromide into contact with a cell or organism.
(IV-2) The method according to Item (IV-1), comprising the steps of:
(1) bringing the viral envelope into contact with the Cas9 protein,
(2) bringing the mixture comprising the viral envelope and the Cas9 protein obtained in step (1) into contact with the surfactant,
(3) bringing the mixture comprising the viral envelope and the Cas9 protein obtained in step (2) into contact with the guide RNA,
(4) bringing the mixture comprising the viral envelope, the Cas9 protein, and the guide RNA obtained in step (3) into contact with at least one positively charged substance selected from the group consisting of protamine sulfate, polyarginine, polylysine, polyethyleneimine, and hexadimethrine bromide, and
(5) bringing the mixture comprising the viral envelope, the Cas9 protein, the guide RNA, and the positively charged substance obtained in step (4) into contact with the cell or organism.
(IV-3) The method according to Item (IV-1), wherein a donor DNA is further brought into contact with the cell or organism in the step.
(IV-4) The method according to Item (IV-2), wherein the mixture comprising the Cas9 protein, the viral envelope and the guide RNA is further brought into contact with a donor DNA in step (3).
(IV-5) The method according to any one of Items (IV-1) to (IV-4), wherein the organism is a non-human organism.
(IV-6) The method according to any one of Items (IV-1) to (IV-4), wherein the cell or organism is a suspension cell.
(IV-7) The method according to any one of Items (IV-1) to (IV-6), wherein the positively charged substance is protamine sulfate.

### BRIEF DESCRIPTION OF DRAWINGS

FIG. 1 shows the test results of Jurkat cells in Test Example 1.

FIG. 2 shows the test results of Jurkat cells in Test Example 2.

FIG. 3 shows the test results of HeLa cells in Test Example 3.

FIG. 4 shows the test results of HeLa cells in Test Example 4.

FIG. 5 shows the test results of Jurkat cells (gRNA target: HPRT1) in Test Example 5.

FIG. 6 shows the test results of K-562 cells (gRNA target: PPIB) in Test Example 5.

FIG. 7 shows the test results of U-937 cells (gRNA target: PPIB) in Test Example 5.

FIG. 8 shows the test results of HeLa cells in Test Example 6. In lane 6, HVJ-E vector suspension composition 6 was added after a 50 μM Scr7¹⁾ solution (knock-in enhancer, NHEJ inhibitor) was added to cells in a plate in an amount of 5 μL/well. ¹⁾ Maruyama T, et al.: Nat Biotechnol, 33: 538-542, 2015

FIG. 9 shows the test results of HeLa cells in Test Example 6.

FIG. 10 shows the test results of primary T-cells (7-week-old mouse) in Test Example 7.

FIG. 11 shows the test results of primary T-cells (9-week-old mouse) in Test Example 7.

FIG. 12 shows the test results of primary T-cells (8-week-old mouse) in Test Example 7.

### DETAILED DESCRIPTION OF EMBODIMENTS

In the present specification, the term “comprise” encompasses the meaning of “essentially consist of” and “consist of.”

In the present specification, the term “encapsulated” means introducing a Cas9 protein and a guide RNA, and optionally a donor DNA, into a viral envelope. The term “HAU” in the present specification indicates an activity of a virus capable of agglutinating 0.5% avian red blood cells. 1 HAU corresponds to approximately 24 million virions.

The genome editing kit of the present disclosure comprises a viral envelope, a Cas9 protein, a surfactant, and at least one positively charged substance selected from the group consisting of protamine sulfate, polyarginine, polylysine, polyethyleneimine, and hexadimethrine bromide.

The genome editing composition of the present disclosure comprises a viral envelope, a Cas9 protein, a guide RNA, and at least one positively charged substance selected from the group consisting of protamine sulfate, polyarginine, polylysine, polyethyleneimine, and hexadimethrine bromide.

The genome editing method and the method for producing a genome-edited cell or organism of the present disclosure (hereinbelow, these methods are sometimes collectively referred to as “the method of the present disclosure”) comprise the step of bringing a viral envelope, a Cas9 protein, a guide RNA, and at least one positively charged substance selected from the group consisting of protamine sulfate, polyarginine, polylysine, polyethyleneimine, and hexadimethrine bromide into contact with the cell or organism.

In the present specification, examples of viral envelopes include envelopes derived from viruses belonging to Retroviridae, Togaviridae, Coronaviridae, Flaviviridae, Paramyxoviridae, Orthomyxoviridae, Bunyaviridae, Rhabdoviridae, Poxviridae, Herpesviridae, Baculoviridae, and Hepadnaviridae. The viral envelope is preferably an envelope derived from Paramyxoviridae virus, more preferably an envelope derived from Sendai virus (HVJ-E), and even more preferably an inactivated HVJ-E.

In the present specification, the term “inactivated” means that a genome is inactivated; inactivated viruses are those that do not undergo genome replication, and lose the ability to proliferate and infect. Examples of virus inactivation methods include UV irradiation, radiation irradiation, a treatment using an alkylating agent, and the like. UV irradiation and a treatment using an alkylating agent are preferable, and a treatment using an alkylating agent is more preferable.

In the present specification, the viral “envelope” means a membrane structure based on a lipid bilayer membrane that surrounds a nucleocapsid present in a specific virus having an envelope.

Examples of Cas9 proteins are not particularly limited, as long as they are used in CRISPR/CAS9 systems. Usable Cas9 proteins include those capable of forming a complex with a guide RNA to bind to a genome DNA target site, and making single- or double-strand breaks at the target site. Wild types and variant forms of Cas9 proteins are both usable. Examples of variant forms include those having a mutation that deactivates one of the two cleavage domains. Cas9 proteins derived from various organisms are known. Information about amino acid sequences of Cas9 proteins and base sequences encoding for the amino acid sequences are registered in public databases such as NCBI. Using known sequence information, a transformant containing a nucleic acid encoding for a Cas9 protein is formed, thus producing a Cas9 protein. As Cas9 proteins, commercially available products, such as Guide-it Recombinant Cas9, Alt-R S.p. Cas9 Nuclease 3NLS, Alt-R S.p. HiFi Cas9 Nuclease 3NLS, Alt-R S.p. Cas9 D10A Nickase 3NLS, GeneArt Platinum Cas9 Nuclease, TrueCut Cas9 Protein v2, Cas9 Nuclease GFP NLS Protein, Alt-R A.s. Cpf1 Nuclease 2NLS, and dCas9 protein NLS can be used.

Examples of guide RNAs (gRNAs) are not particularly limited, as long as they are used in CRISPR/Cas9 systems. Usable guide RNAs include those that bind to a target site of genomic DNA and to a Cas9 protein, thus directing the Cas9 protein to the target site of the genomic DNA.

To bind a Cas9 protein to genomic DNA, the target sequence of the genomic DNA must have a PAM (Proto-spacer Adjacent Motif) sequence immediately after the target sequence.

The guide RNA includes crRNA (CRISPR RNA) (sequence binding to a target site of genomic DNA), and the crRNA complementary binds to a sequence of a non-target strand that does not contain a PAM complementary sequence. In order to bind to the target site, crRNA includes a sequence having an identity of 90%, 93%, 95%, 98%, 99%, or 100% with the sequence of the target site.

The identity of a base sequence can be calculated by a commercially available analysis tool, or an analysis tool available through electric telecommunication lines (the internet). The identity (%) of the base sequence can be determined by the default setting of a program (e.g., BLAST and FASTA) conventionally used in this field.

The guide RNA also includes tracrRNA (trans-activating crRNA) (sequence binding to a Cas9 protein). The guide RNA guides the Cas9 protein to the target, site of genomic DNA because the tracrRNA sequence binds to the Cas9 protein.

The guide RNA usually includes the above-mentioned crRNA and tracrRNA. The guide RNA may be in the form of a single-strand RNA containing crRNA and tracrRNA, or the form of an RNA complex in which crRNA complementary binds to tracrRNA.

Selection of a target sequence and design of a guide RNA can be employed by a known various method. The guide RNA can be prepared according to a known method, such as chemical synthesis (e.g., solid-phase synthesis and liquid-phase synthesis) and biochemical cleavage/recombination.

A donor DNA is used for inserting (knock-in) an intended base sequence into a target site by using HDR (Homology-Directed Repair) occurring at the cleavage site of the Cas9 protein. A donor DNA contains two base sequences (homology arms) having a high identity with the base sequence in the target region, and the base sequence inserted between the arms. The base sequence to be knocked-in is not particularly limited. The strand length of the donor DNA is not particularly limited, and is, for example, between 50 b to 500 kb or 50 b to 5 kb.

As the donor DNA, single-strand DNA and double-strand DNA can both be used. From the viewpoint of genome editing efficiency, the donor DNA is particularly double-strand DNA. As the donor DNA, both linear DNA and circular DNA can be used. The donor DNA is particularly circular DNA because the frequency of unintended mutations, such as random scraping or addition of DNA, in genome editing can be reduced.

The donor DNA can be prepared by a standard method, such as PCR, chemical synthesis (e.g., solid-phase synthesis and liquid-phase synthesis), or biochemical cleavage/recombination.

The surfactant is not particularly limited. Nonionic surfactants, cationic surfactants, amphoteric surfactants, and anionic surfactants can be used without, any particular limitation. In particular, the surfactant is a nonionic surfactant and an amphoteric surfactant. Of these, octylphenol ethoxylate (e.g., Triton (trademark) X-100), nonylphenol ethoxylate (e.g., NP40), 3-[(3-cholamidopropyl)dimethylammonio]-1-propanesulfonate (CHAPS), and octyl glucoside are more preferable, and octylphenol ethoxylate (e.g., Triton X-100) is even more preferable. The surfactants can be used singly, or in a combination of two or more.

As the positively charged substance, at least one member selected from the group consisting of protamine sulfate, polyarginine, polylysine, polyethyleneimine, and hexadimethrine bromide is used. Of these, the positively charged substance is particularly protamine sulfate. The positively charged substances can be used singly, or in a combination of two or more.

The genome editing kit of the present disclosure contains a viral envelope, a Cas9 protein, a surfactant, the positively charged substance mentioned above, and optionally a guide RNA and/or a donor DNA. The kit of the present disclosure may further contain alcohols (for example, ethanol), buffers (for example, phosphate buffered saline (PBS), HEPES buffer solution, Tris hydrochloric-acid buffer solution, or TE buffer solution), cell culture fluid (for example, DMEM medium, RPMI medium), etc.

The components of the kit of the present disclosure may be contained in separate containers, or any two or more of the components may be contained in a single container.

The composition of the present disclosure is in a form such that the Cas9 protein and the guide RNA, and optionally the donor DNA, are encapsulated in the viral envelope, and protamine sulfate is present on the surface of the viral envelope. The mixing ratio and concentration of each component in the composition of the present disclosure can be suitably selected from the range capable of editing genomes. For example, it is possible to use the mixing mass ratio and concentration of the components in the composition obtained by the method of the present disclosure described below.

In addition to the viral envelope, Cas9 protein, guide RNA, positively charged substance, and optionally donor DNA, the composition of the present disclosure may contain water and additives, such as alcohols (for example, ethanol), buffer solutions (for example, phosphate buffered saline, HEPES buffer solution, Tris hydrochloric-acid buffer solution, or TE buffer solution), cell culture fluid (for example, a DMEM medium, a RPMI medium), etc. The concentration of these additives is not particularly limited. The pH of the composition of the present disclosure is particularly 6 to 10.

The method of the present disclosure can be performed under any conditions. The Cas9 protein and guide RNA are encapsulated into the viral envelope simultaneously using the complex of Cas9 protein and guide RNA, or sequentially using the Cas9 protein and guide RNA separately. In at least one embodiment, the Cas9 protein and guide RNA are separately encapsulated as described below.

In at least one embodiment, the method of the present disclosure is performed by a process including the following steps (1) to (5):

(1) bringing the viral envelope into contact with the Cas9 protein,

(2) bringing the mixture comprising the viral envelope and the Cas9 protein obtained in step (1) into contact with the surfactant,

(3) bringing the mixture comprising the viral envelope and the Cas9 protein obtained in step (2) into contact with the guide RNA,

(4) bringing the mixture comprising the viral envelope, the Cas9 protein, and the guide RNA obtained in step (3) into contact with at least one positively charged substance selected from the group consisting of protamine sulfate, polyarginine, polylysine, polyethyleneimine, and hexadimethrine bromide, and

(5) bringing the mixture comprising the viral envelope, the Cas9 protein, the guide RNA, and the positively charged substance obtained in step (4) into contact with a cell or organism.

The amount of the Cas9 protein that is brought into contact with 100 HAU of viral envelope in step (1) is not particularly limited, and is 0.0001 to 2 nmol or 0.0002 to 0.5 nmol. The concentration of the solution containing the viral envelope used in step (1) is not particularly limited, and is, for example, 10 to 100 HAU/μl or 15 to 82 HAU/μl. It is desirable to perform step (1) at a low temperature, and the temperature of step (1) is, for example, 0 to 25° C. or 0 to 4° C. The contact time is usually about 1 sec to 15 minutes. Step (1) allows for the encapsulation of the Cas9 protein in the viral envelope.

When the mixture comprising the viral envelope and the Cas9 protein is brought into contact with the surfactant in step (2), the final concentration of the surfactant is, for example, 0.001 to 0.5 v/v % or 0.02 to 0.2 v/v %. It is desirable to perform step (2) at a low temperature, and the temperature of step (2) is, for example, 0 to 25° C. or 0 to 4° C. The contact time is usually about 1 sec to 15 minutes. Step (2) improves the efficiency of the encapsulation of Cas9 protein, guide RNA, and donor DNA in the viral envelope.

After step (2), a treatment for reducing the concentration of the surfactant may be performed. Examples of the treatment include removal and exchange of a supernatant by centrifugation and dilution by addition of water, a buffer solution, or the like. Centrifugation is performed as follows: after centrifugation, the supernatant is removed, and the resulting precipitate is resuspended in a liquid such as PBS containing no surfactant.

The amount of the guide RNA that is brought into contact with 100 HA U of the viral envelope in step (3) is not particularly limited, and is, for example, 0.0001 to 2 nmol or 0.0002 to 0.5 nmol. It is desirable to perform step (3) at a low temperature, and the temperature of step (3) is, for example, 0 to 25° C. or 0 to 4° C. The contact time is usually about 1 sec to 15 minutes. Step (3) allows for the encapsulation of the guide RNA in the viral envelope.

In the case of HDR, the mixture comprising the viral envelope and the Cas9 protein is further brought into contact with the donor DNA in step (3). The amount of the donor DNA that is brought into contact with 100 HAU of the viral envelope is not particularly limited, and is, for example, 0.0001 to 2 nmol or 0.0002 to 0.5 nmol. In at least one embodiment, the donor DNA is brought into contact with the viral envelope after the contact of the guide RNA. By further bringing the donor DNA into contact with the viral envelope having the Cas9 protein and guide RNA encapsulated therein, the donor DNA can be encapsulated in the viral envelope.

In step (4), when the positively charged substance is brought into contact with the mixture of the viral envelope, Cas9 protein, and guide RNA obtained in step (3), the final concentration of the positively charged substance is, for example, 0.01 to 5,000 μg/ml or 0.1 to 3,000 μg/ml. It is desirable to perform step (4) at a low temperature, and the temperature of step (4) is, for example, 0 to 25° C. or 0 to 4° C. The contact time is usually about 1 sec to 15 minutes. In the addition of the positively charged substance, dilution is performed with phosphate buffered saline (PBS) etc. after step (3), as necessary. Thus, by the contact with the positively charged substance, the viral envelop contains the positively charged substance on its surface, thereby increasing the genome editing efficiency.

Examples of cells that are brought into contact with the mixture comprising the Cas9 protein, viral envelope, guide RNA, and positively charged substance in step (5) include in vitro cultured cells, cells extracted from a living body, cells present in the living body, and the like. The cells may be adhesion cells or suspension cells. Of these, genome editing of suspension cells can be performed with high efficiency by the method of the present disclosure, as compared with the conventional techniques. Usable cells include a wide variety of cells including early cell lines, stem cell lines, fibroblast cell lines, and immunocyte cell lines (e.g., T-cells, macrophage cells), in which introduction is generally difficult. The cells and organisms used in the present disclosure are particularly mammals, and the cells thereof. Examples of mammals include human, monkeys, mice, rats, guinea pigs, hamsters, cows, pigs, sheep, goat, horses, dogs, cats, rabbits, and the like.

The composition of the present disclosure can be brought into contact with a cultured cell or a cell extracted from a living body, for example, by adding the composition of the present disclosure to a cell suspension or a culture supernatant. In this case, the amount of the viral envelope to be added is not particularly limited, and is, for example, 20 to 60 HAU per 5,000 to 100,000 cells. Subsequently, for example, after the cells are incubated at 37° C. for about 1 hour, they are incubated at an optimum temperature for 20 to 74 hours for genome editing. The composition of the present disclosure can be administered to a living body by an in vivo method, or can be systemically administered. An ex vivo method can also be used. In this case, cells of a target individual are extracted by a known method and treated with the composition of the present disclosure, after which the cells are returned to the target individual.

The kit and genome editing composition of the present disclosure allow for highly efficient genome editing using a CRISPR/Cas9 system. The kit and composition also enable highly efficient genome editing of suspension cells, in which genome editing have been conventionally difficult. Further, in the method of the present disclosure, the Cas9 protein and guide RNA are separately encapsulated in the viral envelope according to a certain procedure, thus further increasing the genome editing efficiency.

The present disclosure enables fast, low-cost genome editing in animals and animal cells in which genome editing (knock-out/knock-in) such as genome destruction and repair has been difficult. This allows for the production of disease-model cells and animals; and leads to treatment research using the cells and animals, as well as functional analysis by screening with cancer cells and pluripotential stem cells.

### EXAMPLES

The present invention is detailed below with reference to Examples; however, the Examples explain specific embodiments of the present invention, and do not limit or restrict the scope of the invention disclosed herein. The present invention includes various embodiments based on the concepts disclosed in this specification. The HVJ-E used in the experiments is the HVJ-E packed in “GenomOne—(trademark) Si” (produced by Ishihara Sangyo Kaisha, Ltd.). The concentration of the HVJ-E is 34.1 HAU per μl.

### Test Example 1

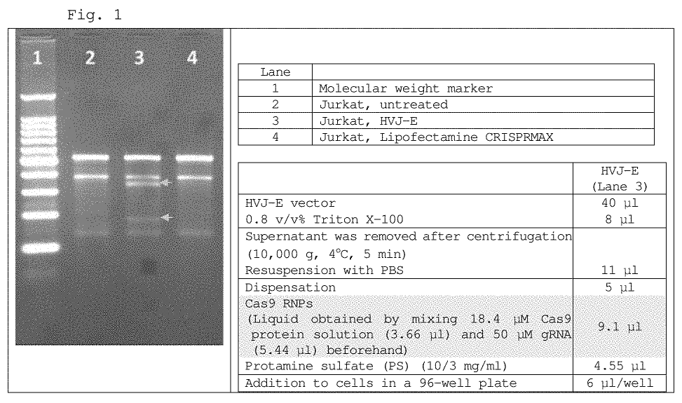

Using a guide RNA (gRNA produced by Dharmacon) targeting Cyclophilin B (PPIB), reagents in the amounts shown in FIG. 1 were used from the top down while the environment was surrounded by ice to maintain the temperature at about 0 to 10° C. First, a Triton X-100 solution (produced by MP Biomedicals, Inc.) was added to HVJ-E, and mixed. The mixture was then centrifuged (100,000 g, 5 minutes, 4° C.) to remove a supernatant, and resuspended in a buffer (phosphate buffered saline (PBS produced by Sigma-Aldrich Co., LLC): components per liter of PBS were NaCl: 8 g, KH₂PO₄: 0.2 g, Na₂HPO₄: 1.15 g, KCl: 0.2 g). Thereafter, the composition was dispensed. After a Cas9/gRNA complex (Cas9 RNPs, a liquid in which a 18.4 μM Cas9 protein solution (3.66 μl) and 50 μM gRNA (5.44 μl) were mixed in advance) was added and mixed, protamine sulfate (PS) (produced by Nacalai Tesque, Inc.) was added to the composition, and the composition was added to cells for genome editing. Genome sequence mismatches generated by editing were detected by the T7 Endonuclease I (produced by NEB) assay (T7E1 assay).

After transfection of a Cas9 protein (produced by Clontech) and guide RNA for genome editing, culturing was performed for two days, and then genomes were extracted. After PCR amplification of approximately 500-bp sequence containing a target region, the purified PCR fragments were reannealed, and reacted with T7E1 that recognizes and cleaves only mismatched DNA. When genomes were edited by the introduced Cas9 protein and guide RNA targeting PPIB, approximately 330-bp and 174-bp fragments were observed by agarose gel electrophoresis (allowed portions in the figure).

FIG. 1 shows the results. A sample based on the above test method corresponds to lane 3. In lane 2, cells were measured without the treatment of reagents such as HVJ-E and Triton X-100. In lane 4, Lipofectamine (trademark) CRISPRMAX (produced by Invitrogen) was used in accordance with the product protocol in place of HVJ-E. In Jurkat cells, which are suspension immune cells that are generally considered difficult to transfect, genome editing did not occur when Lipofectamine CRISPRMAX was used. Genome editing effects were observed in the lane in which the Cas9 protein and the guide RNA were transfected using HVJ-E.

### Test Example 2

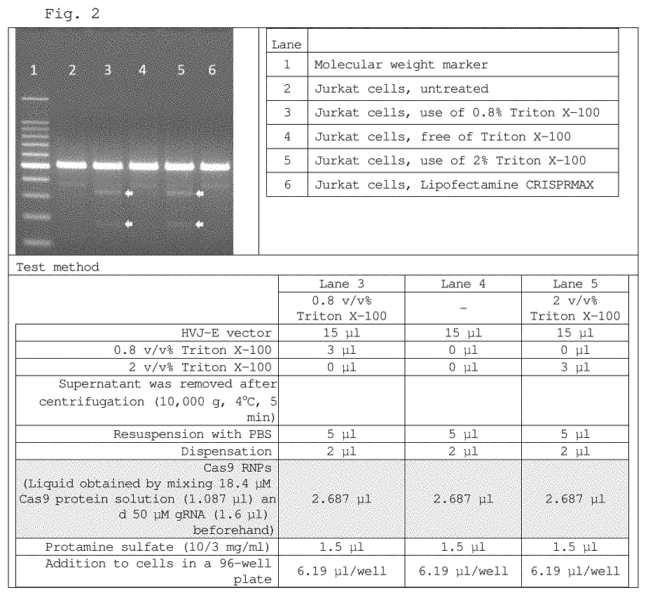

Using reagents in the amounts shown in FIG. 2 from the top down, genome editing was performed in the same manner as in Test Example 1.

FIG. 2 shows the results. Samples based on the above test method correspond to lanes 3 and 5. In lane 2, cells were measured without the treatment of reagents such as HVJ-E and Triton X-100. In lane 4, measurement was conducted in the same manner as in lanes 3 and 5, except that Triton X-100 was not used. In lane 6, CRISPRMAX was used in place of HVJ-E. It was found that in the formation of HVJ-E vector suspension, the addition of a surfactant to HVJ-E allows for the introduction of Cas9 RNPs into cells.

### Test Example 3

Using reagents in the amounts shown in 3 from the top down, genome editing of condition 2 was performed by the same method as in Test Example 1. In condition 1, a Cas9 protein was added and mixed with HVJ-E, and then a Triton X-100 solution in a predetermined concentration was added thereto and mixed, followed by centrifugation, supernatant removal, and resuspension in a buffer (the same as in Test Example 1). Subsequently, after the addition and mixing of a guide RNA, protamine sulfate was added to the composition, and the composition was added to cells.

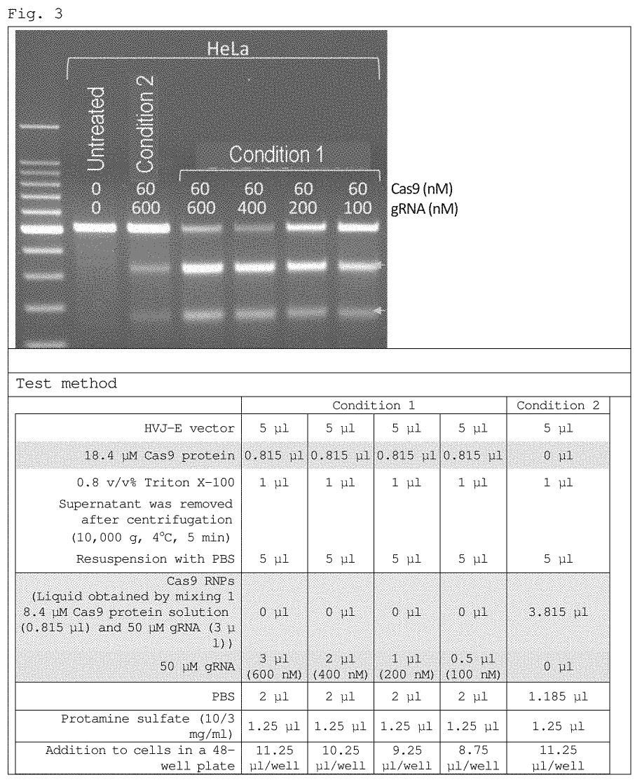

FIG. 3 shows the results. FIG. 3 shows, from the leftmost lane, “molecular-weight marker,” “cells (untreated) measured without the treatment of reagents such as HVJ-E and Triton X-100,” “condition 2,” and “condition 1 containing 600 nM, 400 nM, 200 nM, and 100 nM guide RNAs, in this order.” As is clear from FIG. 3, excellent genome editing effects were attained when the composition obtained by adding Triton X-100 after the addition of a Cas9 protein to HVJ-E, performing centrifugation (100, 000 g, 5 minutes, 4° C.), supernatant removal, and resuspension in a buffer (phosphate buffered saline (PBS), the same as in Test Example 1), and then adding a guide RNA, (PBS), and protamine sulfate to the composition was added to the cells. The production method of condition 1 containing 100 nM guide RNA attained higher genome editing effects than condition 2. Specifically, the production method of condition 1 in which transfection was performed with the HVJ-E vector suspension attained higher genome editing effects than the production method of condition 2 in which the HVJ-E vector suspension was formed using Cas9 RNPs.

### Test Example 4

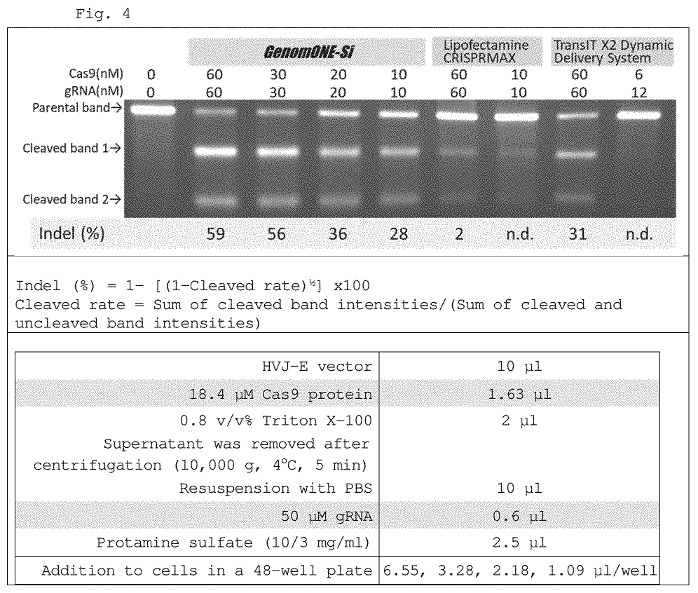

Using reagents in the amounts shown in FIG. 4 from the top down, genome editing was performed. Specifically, after a Cas9 protein was added to HVJ-E, Triton X-100 was added thereto, followed by centrifugation, supernatant removal, and resuspension in a buffer (the same as in Test Example 1). Subsequently, a guide RNA and protamine sulfate were added to the composition, and the composition was added to cells (transfection).

FIG. 4 shows the results. As is clear from FIG. 4, excellent genome editing effects were attained. The cells transfected with the Cas9 protein and guide RNA using an HVJ-E vector attained higher genome editing effects than the cells obtained by using the reagents such as Lipofectamine CRISPRMAX and TransIT-X2 (produced by Mirus Bio LLC).

### Test Example 5

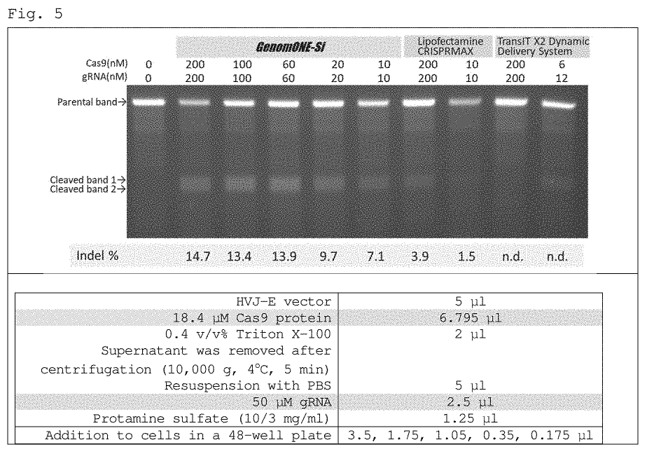

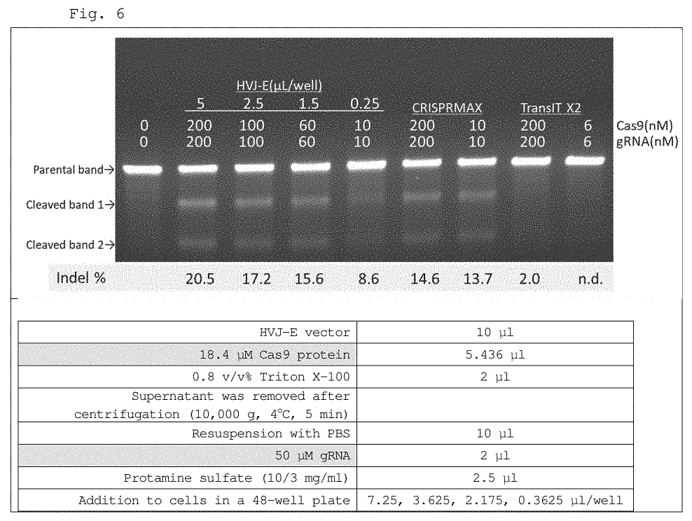

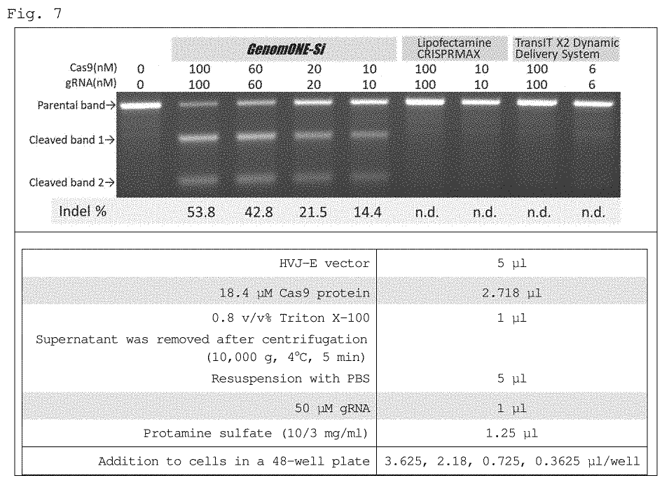

Using reagents in the amounts shown in FIGS. 5 to 7 from the top down, genome editing was performed in the same manner as in condition 1 in Test Example 3. In the experiment shown in FIG. 5, a guide RNA targeting HPRT1 was used.

FIGS. 5 to 7 show the results. In the suspension immune cells of Jurkat cells, K-562 cells, and U-937 cells in which genome editing using the reagents of Lipofectamine CRISPRMAX and TransIT-X2 is difficult, transfection of the Cas9 protein and guide RNA into these cells using the HVJ-E vector attained genome editing effects.

### Test Example 6

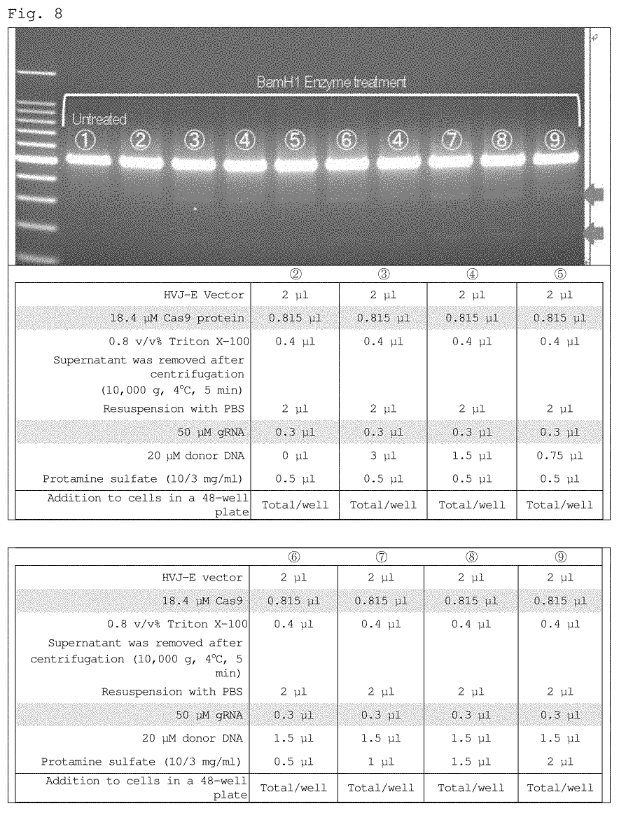

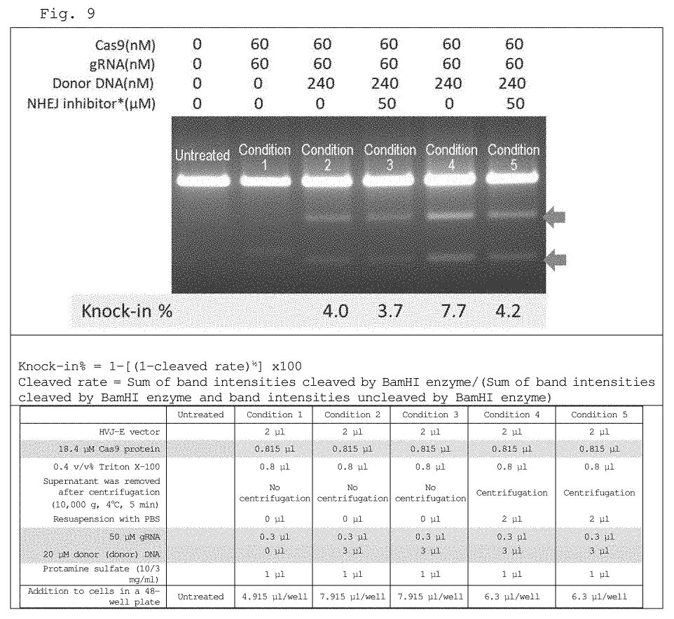

Addition of ssDNA (donor DNA) to the composition of the present disclosure enables recombination (knock-in) genome editing at a guide RNA-targeting site. Reagents in the amounts shown in FIGS. 8 and 9 were used from the top down to perform genome editing. Specifically, an HVJ-E vector suspension containing a donor DNA having a BamHI site (donor DNA in which 30-base homology arms were respectively added to the upstream and the downstream of the base sequence GGATCC to be knocked in, produced by Dharmacon), a Cas9 protein, and a guide RNA was added to cells for transfection; and the transfected cells were cultured in a CO₂ incubator at 37° C. for 2 days, thus extracting genomes from the cells. After PCR amplification of approximately 500-bp sequence containing a target region, the purified PCR fragments were reacted with restriction enzyme BamHI (produced by Takara Bio Inc.). When the target genome for genome editing achieved recombination (knock-in), approximately 330-bp and 174-bp fragments were observed by agarose gel electrophoresis (arrowed portions in the figures).

FIGS. 8 and 9 show the results. Knock-in was observed in HeLa cells. The knock-in effect was enhanced by increasing the amount of protamine sulfate. As compared to the enhancer effect, which is generally considered to enhance the knock-in effect, an increase in the amount of protamine sulfate enhanced the knock-in effect to a greater extent.

### Test Example 7

1. Experiment Using Animals (Extraction of Spleen Cells from Mice)

### 1-1. Animal Control

After animals were brought in, they were grouped per arrival and housed throughout the entire examination period in plastic cages (large) bedded with beta chips. The animals in the cages were not identified. During preliminary breeding, general symptoms were observed to understand the animals' conditions. The animals were freely fed a commercially available, solid complete diet (produced by O.B.S.; MF), and given tap water filtered by active carbon throughout the entire examination period. The cages were exchanged, in principle, three times per week (Monday, Wednesday, Friday).

### 1-2. Test Sample Animals

Kind of animal: Mouse
Name of strain: BALB/cAnNCrlCrlj
Obtained from: Charles River Japan
Sex: Female
Age: 6 weeks old at arrival, 7 weeks old to 9 weeks old when subjected to the test.
   1-3. Extraction of Spleen Cells from Mice and Stimulation of Cells

Spleen cells were extracted from one mouse in each experiment. The extracted spleen cells were adjusted to a concentration of about 1×10⁷ cells/mL, and inoculated on a 10-cm dish. Phorbol 12-myristate 13-acetate (PMA) (final: 500 nM) and Tonomycin (final: 100 μg/mL) were added to the cells, and the cells were stimulated by culturing in a 5% CO₂ incubator at 37° C. (Table 1).

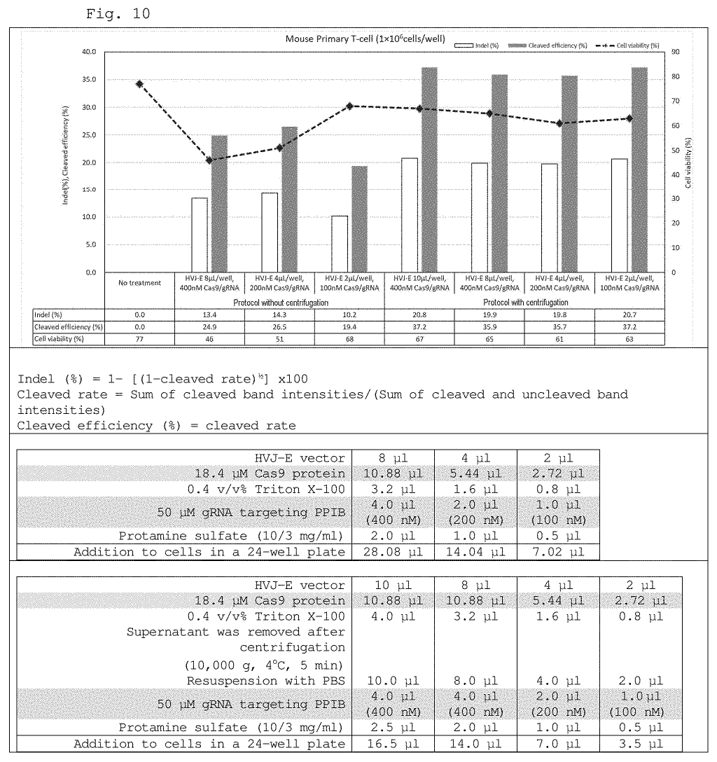

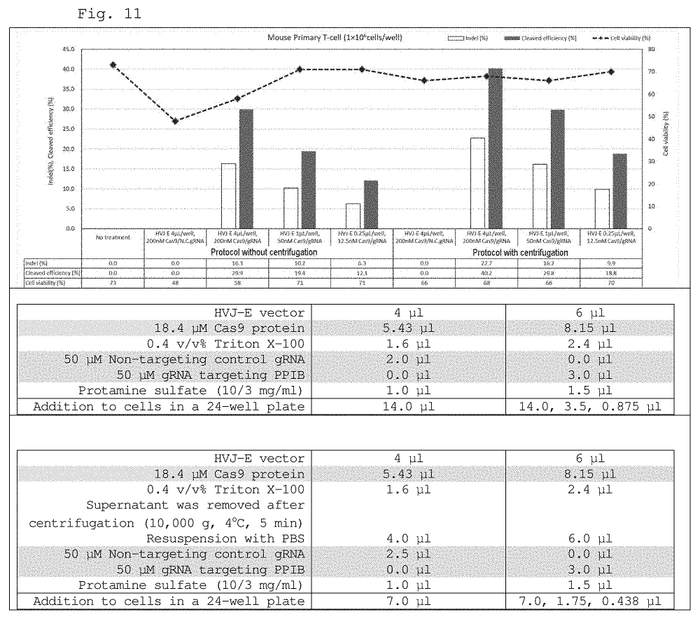

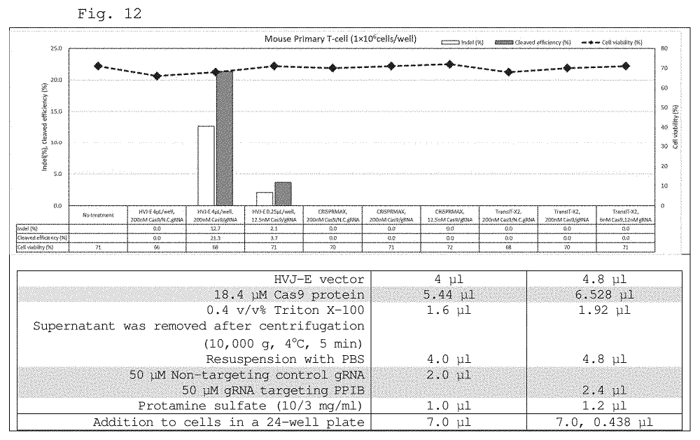

TABLE 1

| | Mouse weight | Number of spleen cells | Stimulation time |
|---|---|---|---|
| | (week age) | (per 10-cm dish) | by culturing |
| FIG. 10 | 19.69 g | 1.0 × 10⁸ cells/10 mL | 22 hr 10 min |
| | (7 weeks old) | | |
| FIG. 11 | 21.34 g | 1.2 × 10⁸ cells/12 mL | 21 hr 00 min |
| | (9 weeks old) | | |
| FIG. 12 | 19.63 g | 1.1 × 10⁸ cells/11 mL | 24 hr 00 min |
| | (8 weeks old) | | |

### 2. Experiment Using Spleen-Derived Cells (Separation of T-Cells)

The stimulated cells derived from the spleen (see Item 1-3 above) were collected to separate T-cells using the Pan T Cell isolation kit II (produced by Miltenyi Biotec; 130-095-130). Using part of the separated T-cells, the PT (Propidium iodide) viability and markers (anti-CD90.2 antibody (CD90.2, PE Anti-Mouse 0.1 mg, clone 53-2.1, Rat IgG2κ) (product of BD Pharmingen; 553005)) were confirmed by a flow cytometer. The same number of cells were used in each experiment condition. The cells were inoculated on a 24-well plate (500 μL/well), and used for an experiment introducing a Cas9 protein and gRNA (Table 2).

TABLE 2

| | Number of T-cells | PI viability | CD90.2 positive |
|---|---|---|---|
| | after separation | (%) | rate (%) |
| FIG. 10 | 1.0 × 10⁷ cells/mL × 1 mL | 78.99% | 95.00% |
| FIG. 11 | 1.2 × 10⁷ cells/mL × 1 mL | 87.40% | 89.75% |
| FIG. 12 | 1.1 × 10⁷ cells/mL × 1 mL | 83.68% | 85.36% |

### 3. Contents of Test

Using reagents in the amounts shown in FIGS. 10 to 12 from the top down, genome editing was performed in the same manner as in Condition 1 in Test Example 3. After culturing, the cell-containing medium was dispensed into each evaluation system to confirm the PI variability and perform the T7E1 assay.

Each cultured cell suspension (50 μl) was diluted 5 to 10 times with 1% BSA/PBS (+), and 5 μL of a 100 μg/mL PI solution was added (final: 1 μg/mL). The PI viability was then measured by a flow cytometer (count number: 10,000).

FIGS. 10 to 12 show the results. As is clear from FIGS. 10 and 11, high genome editing effects were attained in primary T-cells. Further, higher genome editing effects and higher cell variability were attained by centrifugation. Moreover, FIG. 12 indicates that although genome editing did not occur in T-cells when the reagents of Lipofectamine CRISPRMAX and TransIT-X2 were used, genome editing effects were obtained when the Cas9 protein and guide RNA were transfected into T-cells using the HVJ-E vector.

The present application is based on, and claims priority from, JP application Serial Number 2018-093326, filed May 14, 2018, and U.S. provisional application Ser. No. 62/757,901, filed Nov. 9, 2018, the disclosures of which are hereby incorporated by reference herein in its entirety.

1. A genome editing kit comprising a viral envelope, a Cas9 protein, a surfactant, and at least one positively charged substance selected from the group consisting of protamine sulfate, polyarginine, polylysine, polyethyleneimine, and hexadimethrine bromide.

2. The kit according to claim 1, wherein the positively charged substance is protamine sulfate.

3. The kit according to claim 1, further comprising a guide RNA.

4. The kit according to claim 1, further comprising a donor DNA.

5. A genome editing composition comprising a viral envelope, a Cas9 protein, a guide RNA, and at least one positively charged substance selected from the group consisting of protamine sulfate, polyarginine, polylysine, polyethylene imine, and hexadimethrine bromide.

6. The composition according to claim 5, further comprising a donor DNA.

7. A genome editing method comprising the step of bringing a viral envelope, a Cas9 protein, a guide RNA, and at least one positively charged substance selected from the group consisting of protamine sulfate, polyarginine, polylysine, polyethyleneimine, and hexadimethrine bromide into contact with a cell or organism.

8. The method according to claim 7, comprising the steps of:
(1) bringing the viral envelope into contact with the Cas9 protein,
(2) bringing the mixture comprising the viral envelope and the Cas9 protein obtained in step (1) into contact with the surfactant,
(3) bringing the mixture comprising the viral envelope and the Cas9 protein obtained in step (2) into contact with the guide RNA,
(4) bringing the mixture comprising the viral envelope, the Cas9 protein, and the guide RNA obtained in step (3) into contact with at least one positively charged substance selected from the group consisting of protamine sulfate, polyarginine, polylysine, polyethyleneimine, and hexadimethrine bromide, and
(5) bringing the mixture comprising the viral envelope, the Cas9 protein, the guide RNA, and the positively charged substance obtained in step (4) into contact with the cell or organism.

9. The method according to claim 7, wherein a donor DNA is further brought into contact with the cell or organism in the step.

10. The method according to claim 8, wherein the mixture comprising the Cas9 protein, the viral envelope, and the guide RNA is further brought into contact with a donor DNA in step (3).

11. The method according to claim 7, the cell or organism is a suspension cell.

12. A method for producing a genome-edited cell or organism, the method comprising the step of bringing a viral envelope, a Cas9 protein, a guide RNA, and at least one positively charged substance selected from the group consisting of protamine sulfate, polyarginine, polylysine, polyethyleneimine, and hexadimethrine bromide into contact with a cell or organism.
